(19) United States
(12) Patent Application Publication
(10) Pub. No.: US 2026/0295150 A1
(43) Pub. Date: Oct. 1, 2026

(54) SYSTEMS, METHODS, AND DEVICES FOR AMUBLATORY MEDICAMENT ADMINISTRATION

(71) Applicants: Mike Mensinger, San Diego, CA (US); Haripriyan Hampapuram, San Diego, CA (US); Himanshu Patel, San Diego, CA (US); Sean Saint, San Diego, CA (US); Mike Rosinko, Peoria, AZ (US)

(72) Inventors: Mike Mensinger, San Diego, CA (US); Haripriyan Hampapuram, San Diego, CA (US); Himanshu Patel, San Diego, CA (US); Sean Saint, San Diego, CA (US); Mike Rosinko, Peoria, AZ (US)

(73) Assignee: BETA BIONICS, INC., Irvine, CA (US)

(21) Appl. No.: 19/570,342

(22) Filed: Mar. 18, 2026

Related U.S. Application Data
(60) Provisional application No. 63/778,671, filed on Mar. 27, 2025.

(51) Int. Cl. A61M 5/142

(52) U.S. Cl. CPC A61M 5/14244; A61M 2005/14208; A61M 2005/14292; A61M 2202/0007; A61M 2205/3303; A61M 2205/3327; A61M 2205/50; A61M 2230/005; A61M 2230/201

(57) ABSTRACT

A method for administering medicament from a medical device includes receiving, from a sensor, a first biomarker level in a user, determining a basal rate of endogenous glucose production based on one or more biomarker levels, determining, based on the basal rate, an amount of medicament to transfer to the user, transmitting a first instruction to the medical device to deliver the amount of medicament to the user, further receiving, from the sensor, a second biomarker level in the user, adjusting the basal rate based on the first biomarker level and the second biomarker level, adjusting the amount of medicament to transfer to the user based on the adjusted basal rate, and transmitting a second instruction to the medical device to deliver the amount of medicament to the user.

### CROSS-REFERENCE TO RELATED APPLICATIONS

The present application claims the benefit of US Provisional Application No. 63/778,671, filed Mar. 27, 2025, the contents of which are incorporated herein by reference in their entirety.

### FIELD OF THE INVENTION

The present disclosure generally relates to the field of ambulatory medical devices for administering medicaments to patients.

### BACKGROUND

Ambulatory medical devices allow patients the freedom to treat themselves while being mobile. The patient may be tasked with operating the ambulatory medical device. In some cases, the ambulatory medical device is configured to operate without input from the patient. Some ambulatory medical devices are configured to administer medicament responsive to received input from the patient. Many ambulatory medical devices are configured to administer medicament to the patent away from medical supervision. There is a continual need in the art for improvements in ambulatory medical devices to enhance all aspects of their operation.

### SUMMARY

An ambulatory medical device may deliver medicament to a user. The ambulatory medical device determines the amount and rate of medicament to administer over a continuous period of time. The ambulatory medical device may continuously adjust a basal rate of endogenous production in the user, which may be factored to determine the amount of medicament delivered over time.

The basal rate may be subject to continuous adjustment based on various parameters and statistics, including biomarkers collected from the user, the user's basal rate, meal times, and data-derived statistics from biomarkers. In some embodiments, the basal rate may be adjusted upward or downward based on the user's response to medicament administration.

For example, if a user, who has type 2 diabetes, exhibits a blood glucose level that dips below a threshold for more than 5% of a period of time while using the ambulatory medical device, the basal rate may be adjusted downward. Conversely, if the user blood glucose levels dips below the threshold for less than 3% of the period of time, the basal rate may be adjusted upward.

### BRIEF DESCRIPTION OF THE DRAWINGS

FIG. 1 is a schematic of an embodiment of the disclosed ambulatory medical device.

FIG. 2 is an illustration showing a user using an embodiment of the disclosed ambulatory medical device.

FIG. 3 is a blown-up view of a system for delivering medicament into a user using an embodiment of the disclosed ambulatory medical device.

FIG. 4 is a blown-up view of an embodiment of the ambulatory medical device for delivering medicament into a user.

FIG. 5 is a schematic of an embodiment of a system for adjusting a basal rate in an embodiment of the ambulatory medical device.

FIG. 6 is a set of screenshots showing a meal announcement interface.

FIG. 7 is an illustration of a screen that a user may interact with in an embodiment of the disclosed ambulatory medical device.

FIG. 8 is a graph of insulin delivered over a period of a day.

FIG. 9 is a flow diagram of a system for adjusting a basal rate in an embodiment of the disclosed ambulatory medical device.

FIG. 10 is a schematic of a computer system that may perform processing tasks or other computing tasks in various embodiments of the disclosed ambulatory medical device.

### DETAILED DESCRIPTION

The disclosed subject matter is a system for delivering medicaments using an ambulatory medical device. The system determines the amount of medicament to deliver to a user based on input and feedback from the user. In various embodiments, the system may determine an amount of medicament based on biomarker feedback from the user. For example, various algorithms may be used to determine the amount of medicament to deliver to the user. The amount of medicament may be varied continuously and dynamically over time.

The disclosed subject matter delivers medicament to a subject wearing the device. The ambulatory medical device may learn or determine various factors, biomarkers, or physiological metrics for the subject. The ambulatory medical device may adjust based on the physiological metrics to fine-tune the amount or rate of medicament into the subject, and adjust the medicament infusion based on the physiological metric.

In some embodiments, the physiological metric is an endogenous basal rate. Endogenous basal rate refers to the natural, baseline rate of a physiological process within the body, such as insulin production.

In embodiments, the endogenous basal rate may be determined based on biomarkers collected from the subject. For example, the continuous glucose monitor may provide the ambulatory medical device with continuous glucose levels. The ambulatory medical device may further deliver a medicament such as insulin into the subject based on the biofeedback or biomarker response from the subject. Based on this response, the basal rate may be adjusted to learn how much glucose or insulin is naturally produced by the individual. The basal rate may be further adjusted based on biomarker feedback to learn how the basal rate changes in response to various events such as meals, exercise, time of day, stress, etc.

In an exemplary embodiment, the medicament may include insulin. In another exemplary embodiment, the medicament may include glucagon. Insulin is a hormone that regulates blood glucose levels by facilitating glucose uptake into cells. Glucagon is a hormone that raises blood glucose levels by promoting the release of stored glucose from the liver. In various embodiments, the medicament may be delivered automatically by the ambulatory medical device. The term ambulatory, as used herein, describes a device that may be worn by the user and is not connected to any stationary device such as connected to a wall or outlet. The ambulatory medical device allows the user to freely travel and go where they please while the ambulatory medical device treats the patient or user. The ambulatory device may be carried by the user or may be adhered to the patient's body.

In various embodiments, the ambulatory medical device may include an insulin sensitivity factor. The insulin sensitivity factor may be determined using various algorithms and may be adjusted continuously over time. The insulin sensitivity factor, as used herein, may include a gain term that determines the aggressiveness with which the patient is treated with insulin. In various embodiments, the gain term may also be applied to other medicaments besides insulin. In an exemplary embodiment, the ambulatory medical device may determine an amount of unmetabolized insulin in the user. In yet another exemplary embodiment, the ambulatory medical device may determine a basal rate in the user. The basal rate, as used herein, may refer to the rate at which insulin is produced by the user. In yet another exemplary embodiment, the ambulatory medical device may include one or more meal adaptations. For example, the ambulatory medical device may learn to adjust medicament delivery based on meals taken by the user.

The ambulatory medical device may connect to a user to deliver medicament to the user. For example, the ambulatory medical device may be worn by the user administer medicament using a transdermal needle. The ambulatory medical device may include a pump that delivers medicament to the user. In an embodiment, the pump is a lead screw-driven syringe pump that includes a motor connected to a lead screw that converts rotational motion into linear motion. The lead screw may drive a plunger within a syringe to dispense a controlled amount of medicament to a user at a predetermined rate. Another example of a pump may be a peristaltic pump, which moves fluid through a flexible tube using rotating rollers. The ambulatory medical device may further include a controller that determines an amount of medicament and instructs the pump to deliver the medicament to the user. The ambulatory medical device may also include one or more reservoirs for medicament. The controller may determine an amount of medicament to deliver to the user. The ambulatory medical device may continuously modify or adjust the amount of medicament based on time and various sensor inputs from the user. For example, the ambulatory medical device may collect biomarker data from the user and adjust the amount of medicament to be delivered. The ambulatory medical device may also be operated by the user, such as allowing the user to enter meal times or meal breaks and specify the size of a meal.

An exemplary embodiment of the ambulatory medical device includes a corrections controller. The corrections controller may be a control mechanism that calculates small insulin doses based on near-continuous calculations of the deviation of current glucose from a target level. The controller may include a gain term that controls how many units of insulin to deliver per mg/dL that the glucose is above target. The gain term can be considered an insulin sensitivity factor or an aggressiveness term. Additionally, the controller may take into account any insulin that has been delivered but has not yet been metabolized. The insulin amount that has not yet been metabolized is the insulin on board.

The gain term of the ambulatory medical device controls an amount of medicament delivery. For example, the gain term in an insulin-delivering medical device may adjust the insulin sensitivity factor, which determines the amount of medicament delivered per time period to a user. A higher gain term may result in more aggressive medicament delivery, while a lower gain term may result in less aggressive medicament delivery. The gain term may change over time. The ambulatory medical device may collect biomarker data and adapt the gain term based on the biomarker data. For example, the gain term may be modified by the ambulatory medical device based on glucose readings from the user. The ambulatory medical device may vary the gain term based on each user. Further, the gain term may be adjusted based on past delivery data.

The gain term may be initialized based on various parameters such as body mass, total daily dose, and similar factors. In an exemplary embodiment, the gain term may be adapted based on glucose statistics. For example, a target glucose statistic may be determined by evaluating glucose levels at regular time intervals. If glucose at the end of a time period is greater than a conditional threshold, the gain term may be increased by a set percentage. If glucose at the end of the time period is below a conditional threshold, the gain term may be decreased by a set percentage. For example, if the total time that the glucose below 70 mg/dL over the past 24 hours exceeds 5% then the gain term may be decreased, or if the total time that the glucose is below 120 mg/dL is less than 5% it may be increased

Similarly, the gain term may be adjusted based on mean glucose levels. If the mean glucose is greater than a target, such as 150 mg/dL, the gain term may be increased. If the mean glucose is less than the target, the gain term may be decreased. Other statistical methods may also be used to adjust the gain term. For example, both the mean and standard deviation of glucose levels may be used to calculate a lower glucose limit. The lower limit may be determined using the measured mean, standard deviation, and a t-statistic with a significance level of 0.10. If the calculated lower statistic is below a threshold, the gain term may be decreased. If the calculated lower statistic is above the threshold, the gain term may be increased.

The gain term may also be adjusted based on the statistics of medicament delivery. For example, a patient may have a specific total daily insulin dose that is expected, and the gain term may be increased if the actual insulin delivered is below this target. Similarly, in a bi-hormonal system using insulin and glucagon, the insulin gain term may be decreased when glucagon dosing is higher than expected.

The gain term may also be adjusted when glucose is very high. For instance, the gain term may be reduced to prevent abnormally high insulin doses. In an example of use, a system may linearly decrease the gain term with glucose levels starting with full ‘gain term’ at 250 mg/dL and decreasing to half ‘gain term’at 400 mg/dL.

In an exemplary embodiment, the ambulatory medical device may determine insulin on board and adapt various medicament delivery amounts based on the insulin on board. The insulin on board, as used herein, refers to unmetabolized insulin in the patient. When a patient starts it is unclear what their insulin on board is. If the patient's glucose is high, they may have also have high insulin on board to bring that glucose down that was delivered from a previous pump or pen injection. If glucose is high and insulin on board is high and if the system does not know about the insulin on board, the system may over deliver insulin. On start-up, a system may calculate the amount of insulin needed for the current glucose levels and assume that there is that much insulin on board which will reduce the initial corrections dosing until any residual insulin on board is metabolized.

Every patient or user metabolizes insulin at a different rate. Accordingly, the ambulatory medical device may adjust the determination of insulin on board specifically for each user. A determination of the remaining un-metabolized insulin in the patient may be performed using an estimate of insulin t-max, which is the time at which the pharmacodynamic effect of a single insulin dose reaches its maximum level. Alternatively, the calculation may use insulin duration, which represents the total period during which a single dose of insulin affects the patient's glucose levels. These insulin activity parameters may vary from patient to patient. The system may adapt these parameters to the individual patient.

The ambulatory medical device may also factor in a basal rate for the user. Each patient may have a basal rate of endogenous glucose production, which ideally may be compensated for by the basal insulin rate. The nominal basal rate can be estimated and adapted, with instantaneous adjustments made based on glucose levels. For example, a sensor may receive glucose readings from the user and transmit them to the controller. Based on the glucose levels, a basal rate can be estimated. In various embodiments, an initial basal rate may be estimated based on user parameters such as body mass and total daily dose.

In an exemplary embodiment, a nominal basal rate may be adjusted using a method similar to the insulin sensitivity factor adaptation referenced above. For example, glucose levels may be evaluated over a 24-hour period, with readings collected every six hours. Each time a glucose level statistic violates a threshold, the basal rate may be decreased by a certain percentage. Each time a glucose level statistic violates a different threshold, the basal rate may be increased by a certain percentage. For example, if the total time that the glucose below 70 mg/dL over the past 24 hours exceeds 5% then the basal rate may be decreased, or if the total time that the glucose is below 120 mg/dL is less than 5% it may be increased

In an exemplary embodiment, the basal rate may be determined based on the amount of medicament previously delivered. For example, a nominal basal rate may represent the amount of insulin required to counteract a patient's nominal endogenous glucose production (EGP). The EGP rate may correspond to the lowest amount of insulin delivered throughout the day.

Accordingly, the nominal basal rate may be adjusted by monitoring insulin delivery over time. For instance, the basal rate may be set based on insulin delivery during a time period with the lowest insulin delivery, the basal rate may then be further adjusted based on changes in insulin on board and glucose levels during that time period, setting the basal rate to the nominal basal rate.

The adjustments for insulin on board (IOB) may be made by subtracting the IOB at the beginning of the period from the IOB at the end of the period and using this difference to adjust the measured insulin delivery. Similarly, if glucose levels dropped during the period, the measured insulin delivery may be decreased, as it indicates that insulin delivery exceeded the patient's endogenous glucose production. Conversely, if glucose levels increased, the measured insulin delivery may increase.

In embodiments, the system may undo learning when an occlusion is detected. For example, if an occlusion is detected, look back for a period of time and undo learning of the nominal basal rate and the gain term. Examples of the occlusion may include an unannounced meal, a medicament infusion or injection from another source, extreme exertion, and similar events that deviate from the subject's daily routine The look-back period may be fixed or may be based on insulin delivery. For example, occlusions may require 3 units of insulin to detect. Accordingly, the system may look back for the last 3 units of insulin or monitor motor current history to determine when the current increased.

In an exemplary embodiment, the ambulatory medical device includes a meal controller. The meal controller may adjust medicament delivery based on meals taken by the user. For example, a user may use one or more inputs on the ambulatory medical device to indicate that a meal is being taken. The ambulatory medical device may then adjust medicament delivery based on this input. In various embodiments, the ambulatory medical device may learn from past meals and biomarker feedback to adjust the amount of medicament or insulin delivered in response to a meal. For example, if a user exhibits a consistent biomarker reaction to a specific meal over time, the ambulatory medical device may adjust insulin delivery near mealtime based on the user's historical biomarker data.

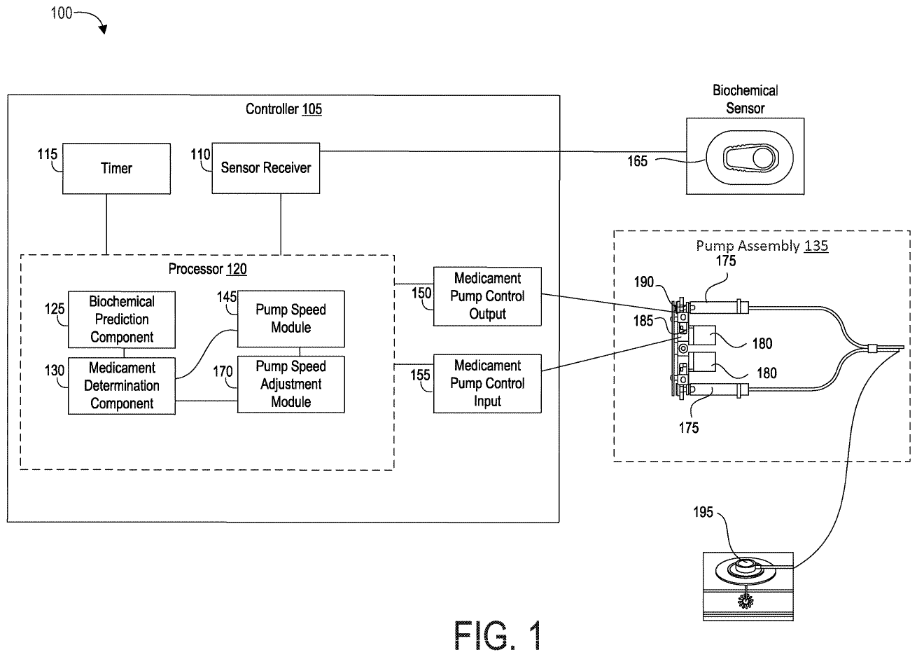

Referring to FIG. 1, FIG. 1 is a schematic 100 of an embodiment of the disclosed ambulatory medical device. The ambulatory medical device may be worn by a user, allowing the user to move freely while the ambulatory medical device delivers a medicament at a prescribed rate. The ambulatory medical device may collect biomarker data from the user to adjust medicament delivery. The ambulatory medical device may be configured to deliver one or more medicaments based on the user's specific parameters. For instance, if the user has type 2 diabetes, an ambulatory medical device can adjust insulin delivery variably based on activity level, meal times, basal rate, and past medicaments. Accordingly, the ambulatory medical device may continuously adjust the amount of medicament delivered to the user based on a variety of parameters.

In the exemplary embodiment of the ambulatory medical device shown in schematic 100, the ambulatory medical device includes a controller 105, a biochemical sensor 165, a pump assembly 135, and a transdermal syringe 195. The controller 105 may collect various data, such as user input, biomarker sensor data from a biochemical sensor 165, and other information, including time and wireless communication data from one or more sources. The biochemical sensor 165 may include one or more sensors that collect biomarker data from the user. For example, the biochemical sensor 165 may include a glucose sensor attached to the user. An example of a glucose sensor may be a continuous glucose monitor (CGM) that measures interstitial glucose levels in real time. The pump assembly 135 may include one or more reservoirs, pumps, and motors configured to deliver medicaments from the reservoirs to the user in response to instructions from the controller 105. The transdermal syringe 195 may attach to the user and provide a connection that allows medicament to be delivered from the medicament pump assembly to the user.

The controller 105 may control medicament delivery to the user and determine modifications in medicament delivery. The controller 105 may receive input from various sources, such as the user, the biochemical sensor 165, and other relevant data sources. The controller 105 may adjust medicament delivery based on a variety of factors, including learning over periods of time from a user's historical readings, such as historical glucose readings. In the exemplary embodiment of the controller 105 shown in schematic 100, the controller 105 may include a timer 115, a sensor receiver 110, a processor 120, a medicament pump control output 150, and a medicament pump control input 155.

The sensor receiver 110 may receive various data from outside the ambulatory medical device. For example, the sensor receiver 110 may receive data from a biochemical sensor 165. Examples of data received from the biochemical sensor 165 may include biomarker data such as blood glucose levels, blood oxygen levels, body temperature, blood insulin levels, blood ketone levels and similar physiological metrics. The sensor receiver 110 may also receive data from other sources. For example, the sensor receiver 110 may receive signals from outside the ambulatory medical device to initiate or modify certain actions. An example of a sensor receiver 110 receiving external signals may include retrieving historical user data from a database server. In another example, the sensor receiver 110 may receive an instruction from a server to perform a specific action. In one instance, the sensor receiver 110 may start or stop a treatment based on an instruction received from a server.

In various embodiments, the sensor receiver 110 may be wired or wireless. Sensor receiver 110 may connect to one or more biochemical sensors or other sensors collecting biomarker information from the user. In various embodiments, the sensor receiver 110 may include both wired and wireless connections to various sensors on the user, as well as collect data or instructions from external sources. In some embodiments, sensor receiver 110 may be configured to send signals as well as receive them. For example, sensor receiver 110 may be an antenna configured to transmit data from the ambulatory medical device to a server. For instance, the ambulatory medical device may be configured to transmit medical information to a server used by a medical practitioner to evaluate the user.

In one example, the sensor receiver 110 is an antenna that is capable of sending and receiving data. The antenna in the ambulatory medical device may use Bluetooth to send and receive data from other devices. The ambulatory medical device may use other forms of wireless communication, including but not limited to Wi-Fi, LTE, and NFC. In various embodiments, the wireless communication antenna that transmits and receives wireless data is placed as far as practicable from components and materials that may interfere with the wireless signal within the ambulatory medical device. In one embodiment, the ambulatory medical device comprises a metal housing connected to a non-conductive bezel. The wireless communication antenna is placed adjacent to the non-conductive bezel such that interference with the wireless signal from the metal housing is diminished compared to other locations inside the ambulatory medical device.

The timer 115 on the ambulatory medical device may track various aspects of medicament treatment. For example, the timer 115 or the processor 120 may collect biomarker statistics from the user. In one instance, the processor 120 may collect blood glucose levels from the user every six hours. In another instance, the processor 120 may calculate an average glucose level over a six-hour period. The timer 115 may be used to schedule the collection of biomarker events in various embodiments. The timer 115 may also be used to regulate the timing of medicament delivery. For instance, the processor 120 may issue an instruction for the pump to deliver a specified amount of medicament over a designated time period. The timer 115 may be utilized to deliver medicament at an appropriate rate. In various embodiments, the processor 120 may instruct the pump to continuously update the rate of medicament delivery over time. Accordingly, the timer 115 may be used to determine how the rate changes. In various embodiments, the processor 120 may adjust a gain term based on statistics collected using the timer 115. Additionally, the processor 120 may adjust a gain term based on an anticipated mealtime, which may also be determined partially by the timer 115.

The medicament pump control output 150 may receive instructions from the processor 120 to control the output of the medicament pump 175. For example, the processor 120 may determine a rate to administer medicament and transmit an instruction to the medicament pump control output 150 to deliver medicament at that rate. The medicament pump control output 150 may then transmit the instruction to medicament pump assembly via an electric signal to pump at the desired rate. In various embodiments, the processor 120 may transmit an instruction to deliver a specific volume of medicament to the user. The medicament pump control output 150 may transmit an instruction to deliver the volume of medicament at a default rate. The medicament pump control output 150 may further transmit an instruction to stop medicament delivery once the specified volume has been administered.

The medicament pump control output 150 and the medicament pump control input 155 may send and receive data to and from the pump assembly 135, respectively. The pump assembly 135 may be connected to the controller 105 via a wired or wireless connection. If the pump assembly 135 is connected via a wireless connection, it may include a transceiver capable of sending and receiving signals to and from the controller 105.

The medicament pump control input 155 may receive signals from the pump assembly 135. In an embodiment, the medicament pump control input 155 may receive a signal from the pump assembly 135 that tabulates the amount or volume of medicament delivered to the user. Accordingly, data collected by the medicament pump control input 155 may account for any differences between the desired volume of medicament to be administered and the actual amount administered. In an exemplary embodiment, a magnetic encoder on the pump assembly 135 may record the number of times gears spin within the pump assembly 135, where the gears control the amount of medicament delivered. The medicament pump control input 155 may transmit data from the magnetic encoder to the processor 120 to determine the actual amount of medicament administered. The processor 120 can adjust the rate or volume of medication administered based on data from the medicament pump control input 155.

The processor 120 may collect data from outside the ambulatory medical device, control various functions of the ambulatory medical device, determine treatment administered by the ambulatory medical device, and communicate with or send instructions to other devices. The processor 120 may be a computing system capable of performing various computing tasks, such as determining an amount of medicament to be administered to a user, receiving data and instructions from outside the ambulatory medical device, and similar operations. In the embodiment of the processor 120 shown in the schematic 100, the processor 120 may include a biochemical prediction component 125, a medicament determination component 130, a motor speed module 145, and a pump speed adjustment module 170.

The biochemical prediction component 125 may determine, estimate or predict various biological processes in the user. For example, the biochemical prediction component 125 may be configured to determine an insulin t-max level in the user. The t-max, as used herein, is the time at which the insulin level, or other chemical, reaches its maximum concentration in the user. The biochemical prediction component 125 may also be configured to determine a basal rate in the user. For example, the biochemical prediction component 125 may predict the rate at which insulin is produced in the user at various times of the day. For instance, the biochemical prediction component 125 may predict a basal rate in the morning, afternoon, and evening for a user. The biochemical prediction component 125 may further determine various medical processes occurring in the user based on data collected by the controller 105. For example, if the biochemical sensor 165 sends data indicating an increased temperature in the user, the biochemical prediction component 125 may predict that the user's metabolic rate is increasing and respond by adjusting medicament delivery.

The medicament determination component 130 may determine an amount of medicament or rate of medicament delivery to administer treatment to the user. For example, the medicament determination component 130 may determine a default rate to administer to the user. In some embodiments, the medicament determination component 130 may use one or more biomarker sensor readings collected from the biochemical sensor 165 to adjust the medicament treatment rate. For example, if the biochemical sensor 165 collects data indicating that the user's blood glucose level is high, the medicament determination component 130 may increase the rate of medicament delivery to address the elevated glucose level. For instance, the medicament determination component 130 may increase the rate of insulin delivered to the user. In some embodiments, the medicament determination component 130 may determine a dynamic rate of medicament delivery that continuously changes over time. For example, a user may have a historical reaction to meals. In one instance, data collected over a long period of time may indicate a blood glucose response to a lunch meal. Accordingly, the medicament determination component 130 may determine an insulin delivery rate that continuously adjusts for a period of time after a meal based on historical data.

In further examples, the medicament determination component 130 may adjust a medicament delivery rate based on the t-max values determined by biochemical prediction component 125. For instance, medicament determination component 130 may lower the medicament delivery rate at t-max and gradually increase the medicament delivery rate afterward. The medicament determination component 130 may adjust the medicament delivery rate before and after t-max periods. In another example, the medicament determination component 130 may adjust medicament delivery based on the basal rate determined by the biochemical prediction component 125. For example, the biochemical prediction component 125 may determine a basal rate of insulin production in the body of the user. The medicament determination component 130 may subtract the predicted insulin production, as determined by the basal rate, from the calculated medicament delivery rate.

The motor speed module 145 in the processor 120 converts the output from the medicament determination component 130 into a pump speed that delivers medicament at the rate and amount determined by the medicament determination component 130. For example, the medicament determination component 130 may specify a rate of medicament administration. The motor speed module 145 may convert that rate into a pump speed and transmit a signal to a motor that operates the pump. The motor speed module 145 may determine an instruction that causes the motor to spin and operate the pump at a set speed or to advance a lead screw to deliver a set volume. In some embodiments, the medicament determination component 130 may determine a volume of medicament to administer to a user. Accordingly, the motor speed module 145 may send a signal that operates the pump for a specified time to deliver the determined volume of medicament. In some embodiments, the medicament determination component 130 may determine a rate of medicament administration over a period of time where the rate changes dynamically. The motor speed module 145 may send an instruction that causes the pump to administer medicament at a dynamically changing rate over time.

The pump speed adjustment module 170 may adjust the speed of the motor speed module 145 based on various data. For instance, data collected via the medicament pump control input 155 may indicate a difference between the actual medicament delivered and the determined medicament delivery. Accordingly, the pump speed adjustment module 170 may adjust the rate of medicament delivery based on this difference.

The biochemical sensor 165 may be a sensor that collects various biomarker data from the user. For example, the biochemical sensor 165 may collect interstitial glucose levels in the user. An example of a blood glucose sensor is a continuous glucose monitor (CGM), such as the Dexcom G7 or the FreeStyle Libre, which continuously measures interstitial glucose levels and transmits data in real time. Other biochemical sensors that may be used for diabetic patients include ketone sensors, which monitor blood ketone levels to detect diabetic ketoacidosis (DKA), and lactate sensors, which measure blood lactate levels as an indicator of metabolic stress. The biochemical sensor 165 may transmit sensor data to the controller 105. In an embodiment, the biochemical sensor 165 may include an insulin sensor.

In some embodiments, the biochemical sensor 165 may transmit data through a wired connection. In other embodiments, the biochemical sensor 165 may transmit data wirelessly. In some embodiments, the biochemical sensor 165 may also receive data instructions. For example, the biochemical sensor 165 may modify one or more parameters in sensor collection based on an instruction received from the controller 105. For instance, the controller 105 may instruct the biochemical sensor 165 to collect data from additional biomarkers. As an example, the controller 105 may instruct the biochemical sensor 165 to collect body temperature data in addition to blood glucose levels. The controller 105 could also direct the biochemical sensor 165 to save energy by collecting biomarker data less frequently.

The pump assembly 135 is an apparatus capable of holding medicament and pumping it to administer the medicament to the user. The pump assembly 135 may include reservoirs, pumps, motors, and connections between the motors and pumps to operate the pumps. In the embodiment of the pump assembly 135 shown in schematic 100, the pump assembly 135 includes a medicament pump 175, DC motors 180, gears 185 connecting the DC motors 180 to the medicament pump 175, and magnetic encoders 190 to record the amount of medicament administered.

The pump assembly 135 may be connected to the user via a transdermal syringe 195. The transdermal syringe 195 may be configured to inject medicament into fatty tissue below the skin of the user. In some embodiments, the pump assembly 135 may pump a medicament through one or more tubes to the transdermal syringe 195. In embodiments where the pump assembly 135 carries more than one type of medicament, it may be connected to multiple transdermal syringes.

In an example of use, the processor 120 may transmit an instruction to the pump assembly 135 to deliver insulin at a specified rate to the user. In another example, the pump assembly 135 may pump insulin at the instructed rate through tubing to the transdermal syringe 195, where the medicament enters the user. In some embodiments, the pump assembly 135 may be connected to the user in alternate ways. For example, in addition to the transdermal syringe 195, the pump assembly 135 may administer medicament through other delivery methods.

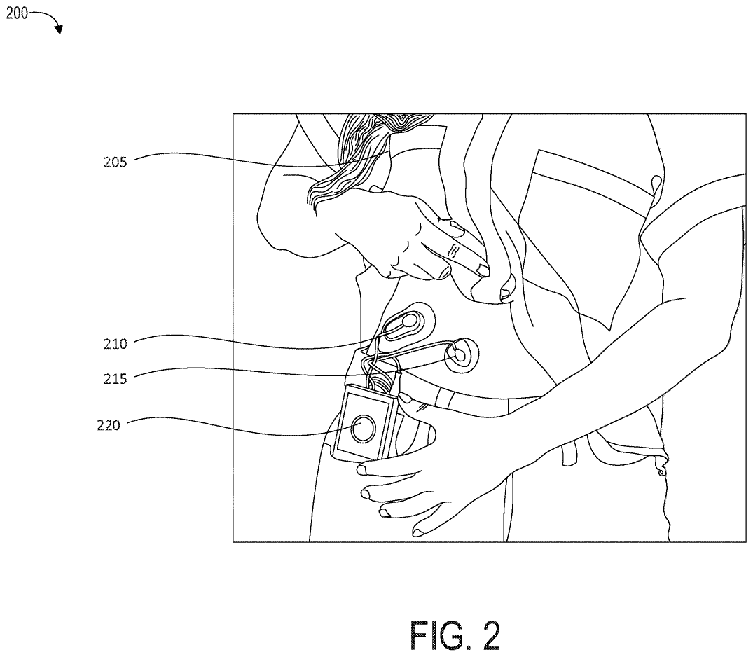

Referring to FIG. 2, FIG. 2 is an illustration 200 showing the user 205 using an embodiment of the disclosed ambulatory medical device 220. The ambulatory medical device 220 may attach to the user 205, allowing the user 205 to walk freely and move to various locations, including outside a medical setting. The ambulatory medical device 220 may be operated by the user 205 and/or by a medical practitioner.

In an example of use, a medical practitioner may configure ambulatory medical device 220 with initial parameters, and the user 205 may operate the ambulatory medical device 220 thereafter. The ambulatory medical device 220 may include one or more input options. Further, the ambulatory medical device 220 may include a processor, a medicament, and a means to administer the medicament to a user 205. In some embodiments, the ambulatory medical device 220 may include a biochemical sensor 210. The biochemical sensor 210 may attach to a user 205 at various locations. As shown in the illustration 200, the biochemical sensor 210 is attached to the user 205 at the side of the abdomen.

The ambulatory medical device 220 may be configured to administer medicaments through a transdermal syringe 215, which is attached to the user 205. Accordingly, the medicament in the transdermal device may be pumped through tubing and delivered into the user 205 via the transdermal syringe 215.

The ambulatory medical device 220 may include various options for the user 205 to input data or operate the ambulatory medical device 220. For example, the ambulatory medical device 220 may include a touchscreen display that enables the user 205 to modify settings or operate the ambulatory medical device 220. For instance, the user 205 may inform the ambulatory medical device 220 when they are about to have a meal. Additionally, the user 205 may provide information regarding the size of the meal. For example, the user 205 may input whether the meal is small, medium, or large. Another embodiment is for the user to indicate a relative size of a meal using a slider which indicates the relative size of a meal for example 50% to 150% of a typical meal. The ambulatory medical device 220 may adjust medicament delivery based on parameters and input from the user 205.

The ambulatory medical device 220 may also learn over time based on data collected from the biochemical sensor 210 and adjust medicament delivery accordingly. For example, the ambulatory medical device 220 may be configured to collect blood glucose levels from the user 205 at various times of day and, based on these levels, determine the basal rate of insulin production in the user 205. The ambulatory medical device 220 may then adjust the insulin delivery rate throughout the day based on learned and collected data.

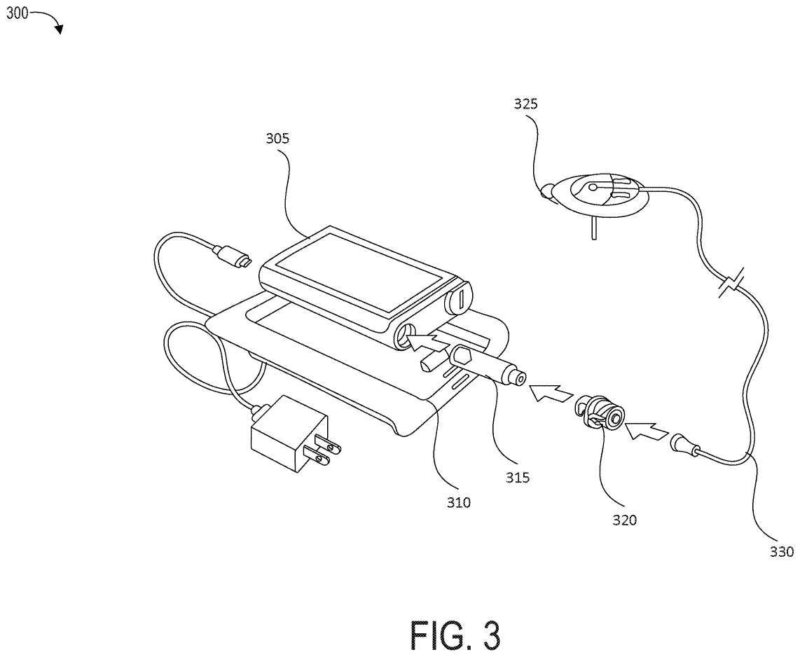

Referring to FIG. 3, FIG. 3 is a blown-up view of a system 300 for infusing medicament into a user using an embodiment of the disclosed ambulatory medical device 220. The system 300 may include various components, including a controller 305, a cartridge 315, a connection adapter 320, flexible tubing 330, and an infusion set 325. In certain embodiments, the controller 305 may be designed to be positioned in a base 310 when it is not being utilized by the user 205. The cartridge 315 may be configured to hold various medicaments in liquid form. In some embodiments, the ambulatory medical device 220 may be charged via a wall charger that connects to it.

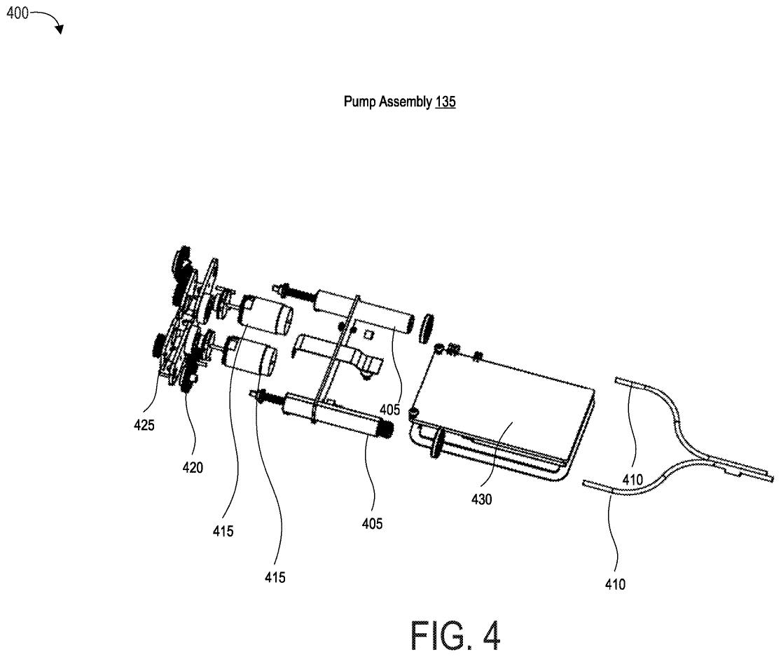

Referring to FIG. 4, FIG. 4 is a blown-up view 400 of an embodiment of the ambulatory medical device 220 for infusing medicament into a user. The ambulatory medical device 220 may comprise a variety of designs and components. The embodiment shown in the view 400 includes a controller 430, one or more cartridges 405, where the cartridges 405 include a pump, and one or more motors 415, where the motors 415 are capable of spinning gears 420. Additionally, the view 400 includes a magnetic encoder 425, which records the spinning of gears 420 to determine the amount of medicament that was pumped.

In some embodiments, the gears 420 may be connected to the cartridges 405, such that spinning the gears 420 operates the pump in the cartridges 405. When the pump is activated, the liquid held in the cartridges 405 may be pumped through tubing 410 and delivered to the user 205.

As shown in the view 400, the embodiment of the ambulatory medical device 220 includes two cartridges 405. In some embodiments, the cartridges 405 may hold different types of medicaments. In other embodiments, the cartridges 405 may hold the same type of medicament. Each cartridge 405 is connected to a separate tubing 410, which delivers medicament to the user 205. Instructions from the controller 430 may cause the motors 415 to spin, which in turn causes the gears 420 to spin, operating the pumps and delivering medicament from the cartridges 405 through the tubing 410 and into the user 205.

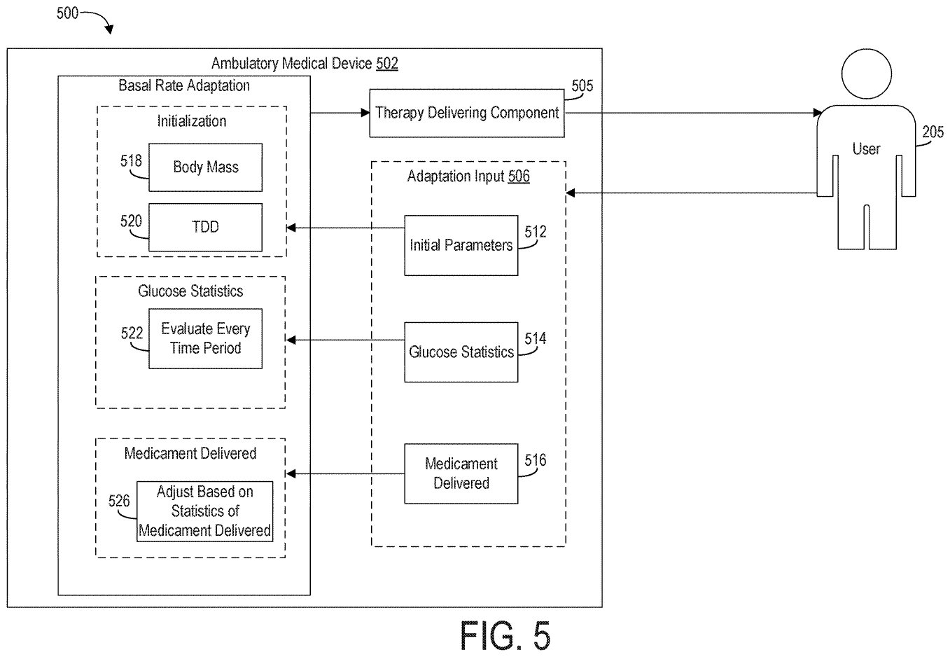

Referring to FIG. 5, FIG. 5 is a schematic 500 of an embodiment of a system for adjusting an endogenous basal rate in an embodiment of the ambulatory medical device 220. The endogenous basal rate may be used to determine the amount of insulin or other medicament delivered per step from the medical device 220.

A step may be a unit of time or another defined interval that represents a segment of time. The ambulatory medical device 220 can deliver a volume of medication over time in multiple steps. The determination of a basal rate may help determine the amount of medicament delivered per step. For example, the ambulatory medical device 220 may subtract the basal rate of endogenous medicament production from an amount of medicament needed. For instance, a subject may require 0.8 units of insulin per hour and have a basal rate of 0.3 units insulin per hour. Accordingly, the ambulatory medical device 220 may deliver insulin at a rate of 0.5 units per hour.

The basal rate may be determined in the basal rate adaptation box in the schematic 500 based on an adaptation input 506. Some examples of the adaptation input 506, as shown in the schematic 500, includes initial parameters 512, glucose statistics 514, and medicament delivered 516.

The adaptation input 506 may be collected as initial parameters and input by medical practitioners. Additionally, the adaptation input 506 may be entered directly and explicitly by the user 205. In some embodiments, the adaptation input 506 may be collected by one or more biochemical sensors 210 attached to the user 205. The adaptation input 506 may also be derived from a combination of these sources. Further, the adaptation input 506 may be retrieved from historical data stored in a database, either on or off the ambulatory medical device 502.

Examples of the initialization parameters 512 include body mass 518 and total daily dose 520. These parameters may be input directly by a medical practitioner or by the user 205. Some initialization parameters 512 may also be collected by a sensor, such as initial biomarkers like blood glucose readings or other biomarker data. The glucose statistics 514 may include various statistics derived from biochemical or biomarker readings of the user 205's blood glucose levels. For example, blood glucose statistics determined from an evaluation every time period 522 may be used to adjust the basal rate.

In glucose statistics, blood glucose levels are measured at regular intervals for each period 522. The time period could be six hours. At the end of six hours, the basal rate is adjusted based on the blood glucose measurement.

For example, if the blood glucose measurement is below a target value for greater than a set percentage of time, the determined basal rate is lowered by a percentage. In one instance of the example, the set percentage of time may be 5%. In the example, if the blood glucose measurement drops below the target value for more than 5% of the total time, the condition to lower the basal rage is satisfied. Likewise, if blood glucose is below the target value for less than a set percentage of time, the basal rate is increased by a set percentage. Once the basal is adjusted, the amount of insulin delivered per step is adjusted based on the basal rate.

In yet another embodiment, the basal rate may be adjusted by the medicament delivered 516. For example, the basal rate may be adjusted based on statistics of the medicament delivered 526. Using insulin as an example, a nominal basal rate may be determined to be the amount of insulin to treat the patient's nominal endogenous glucose production, or EGP. The nominal basal rate, as determined by the patient's nominal endogenous glucose production, can correspond to the least amount of insulin delivered throughout the day. Accordingly, the basal rate can be adjusted based on the insulin delivered throughout the day. For instance, insulin delivered can be recorded for various periods throughout a day. The period with the least amount of insulin delivered can be interpreted as the nominal basal rate.

In various embodiments, insulin on board, or IOB, is determined for a subject. The insulin on board is an estimated amount of insulin that is unmetabolized in the subject's system. The determination of the basal rate is adjusted based on the IOB. For example, the determination of the period with the lowest amount of insulin may be adjusted based on the insulin on board. In this example, the IOB at the start of the period is subtracted from the IOB at the end of the period. The resulting IOB difference is then subtracted from the insulin delivered for each period.

The processor 120 uses the basal rate to determine how much medicament to deliver to the user 205 over time. The medicament may be determined by the medicament determination component 130, whereby an instruction is passed to the therapy delivering component 505, such as the pump assembly 135, which administers the medicament to the user 205.

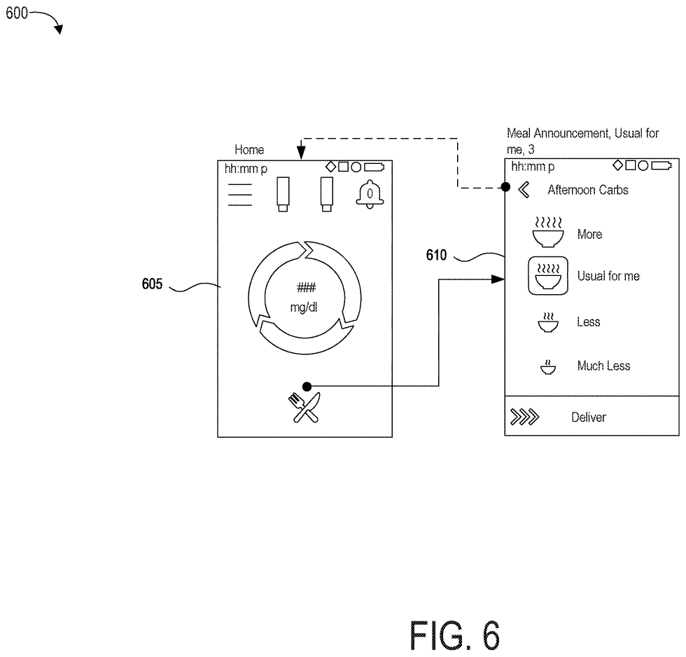

Referring to FIG. 6, FIG. 6 is a set of screenshots 600 showing a meal announcement interface. The ambulatory medical device may include one or more interfaces that allow a user to announce a meal, snack, or other food intake before ingestion. The meal announcement interface may inform the ambulatory medical device 220 that the user's blood glucose level is likely to increase due to the meal.

In an insulin-only system, the ambulatory medical device 220 may increase the rate of insulin infusion in response to the meal announcement. In a bi-hormonal system, which delivers both insulin and glucagon, the ambulatory medical device 220 may adjust both insulin and glucagon delivery based on the expected glucose response. For example, insulin infusion may be increased to compensate for the anticipated rise in blood glucose, while glucagon delivery may be reduced or suspended to prevent unnecessary counteraction.

A first screen 605 may include a user interface that allows a user of the ambulatory medical device to inform the medical device that they are having a meal. As shown in the first screen 605, the user interface includes an icon that enables the user to notify the ambulatory medical device that a meal is imminent.

In various embodiments, selecting the icon may bring up a second screen 610 that allows the user to specify one or more parameters of their meal. For example, as shown in the second screen 610, the user may have the option of selecting a meal size labeled as “more,” “usual for me,” “less,” or “much less.” These options may correspond to different meal sizes, and the ambulatory medical device may adjust a medicament infusion rate accordingly.

In some embodiments, basal rate adjustment may be suspended following a meal announcement. For example, statistics used to measure the lowest insulin level may be obscured based on the meal selection. Accordingly, the ambulatory medical device may pause any determination, adjustment, or statistical analysis related to basal rate calculation for a period of time after a meal announcement.

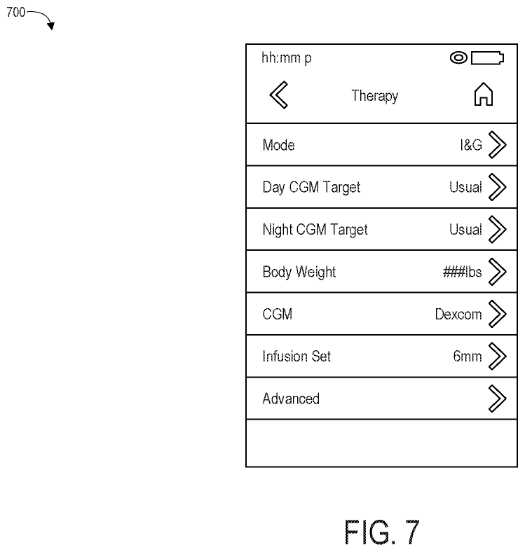

Referring to FIG. 7, FIG. 7 is an illustration of a screen 700 that a user may interact with in an embodiment of the disclosed ambulatory medical device 220. The ambulatory medical device 220 may include multiple input options for the user 205. These input options may allow the user 205 to modify various parameters that affect the determination of medicament delivery, the amount of medicament administered, and the timing of medicament delivery.

Some parameters may affect the determination of the basal rate for the user 205. Other parameters may influence when medicament is delivered and how much variability exists in delivery over a given time period. Examples of inputs that the user 205 may enter into the ambulatory medical device 220 include day and night continuous glucose monitor targets. For example, the user 205 may enter a daytime glucose target of 100-130 mg/dL and a nighttime glucose target of 90-120 mg/dL.

Additionally, the user 205 may input their body weight, which may affect the initial basal rate. Other inputs may be related to hardware settings, such as the mode selection, which determines the types of hormones used for treatment. In the mode shown in the screen 700, the ambulatory medical device 220 is configured to operate with both insulin and glucagon.

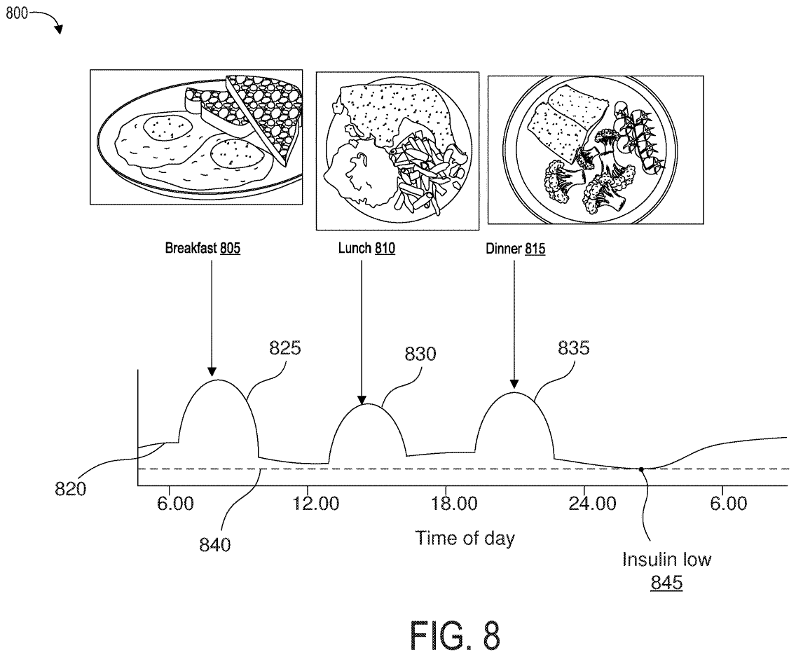

Referring to FIG. 8, FIG. 8 is a graph 800 of insulin delivered over a period of a day. The graph 800 shows bolus additions in response to meals that a user may ingest throughout the day. For example, a user may typically have three meals in a day. Accordingly, the graph 800 of insulin delivered over a day would show three distinct spikes in insulin delivery, corresponding to expected increases in the user's blood glucose levels.

The insulin line 820 represents the insulin delivery rate from the ambulatory medical device 220. As shown by the insulin line 820, there are three bolus additions: a first bolus addition 825, corresponding to breakfast 805; a second bolus addition 830, corresponding to lunch 810; and a third bolus addition 835, corresponding to dinner 815.

In various embodiments, bolus additions may be disregarded in the determination or adjustments made to the basal rate. For example, when the basal rate is determined or adjusted based on the percentage of time it remains below a threshold, the period during which a bolus addition occurs may be excluded from that calculation.

As shown in the graph 800, the nominal basal rate 840 approximates the amount of insulin delivered outside of the periods of bolus additions. This may allow for a more precise determination of the basal rate, which in turn enables more accurate insulin delivery across different time periods.

For example, during a meal, the determination of how much insulin to provide to the user both during and after the meal may be more precise by factoring in the nominal basal rate 840. By isolating the insulin delivered in response to the meal from the nominal basal rate 840, the insulin dosage may better correspond to the size of the meal.

In embodiments, the basal rate is adjusted based on medicament delivery. For example, the nominal basal rate may be determined to correspond to the lowest amount of insulin delivered over a 24-hour period. In the graph 800, the lowest insulin level is indicated by point 845. Accordingly, the nominal basal rate 840 may be adjusted to the insulin level at point 845.

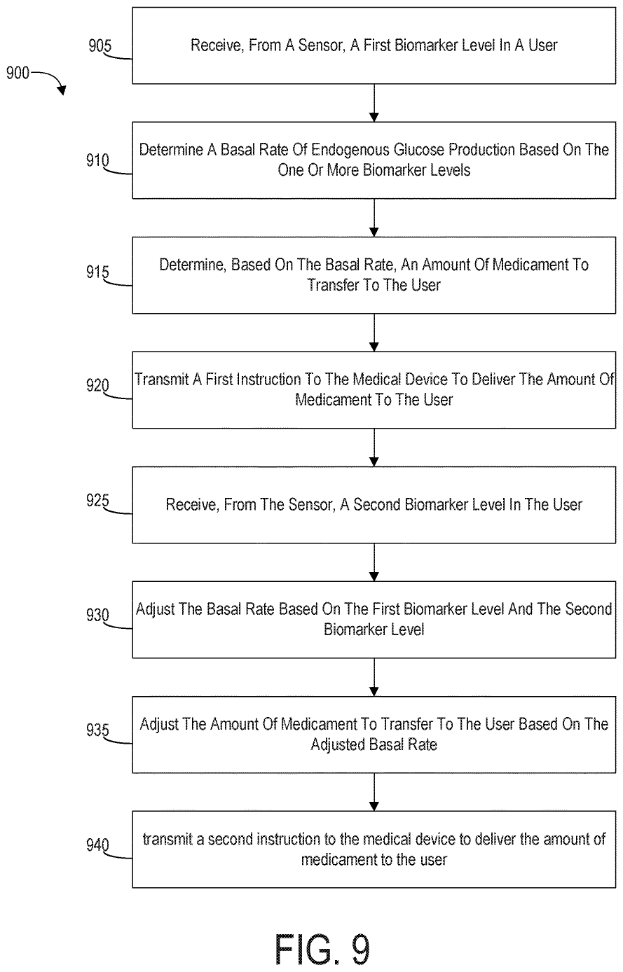

Referring to FIG. 9, FIG. 9 is a flow diagram 900 of a system for adjusting a basal rate in an embodiment of the disclosed ambulatory medical device 220. The basal rate allows for more precise determination of the amount of insulin or other medicament to be infused or injected into the subject. In embodiments, the medicament is insulin and the basal rate is the nominal basal rate of endogenous insulin production in the subject. The basal rate may be determined in various ways. For example, over a period of time, the basal rate may be adjusted based on the medicament infused or added from the ambulatory medical device to 20. For instance, over a 24-hour period, the nominal basal rate of endogenous insulin production in the subject may be adjusted to correspond with the lowest amount of insulin delivered over a time period within that day. For example, the lowest insulin delivered over a 3-hour period may be determined to correspond to the basal rate of endogenous insulin production in the subject.

In another example, the basal rate is determined based on the percentage of time that the insulin delivery rate falls below a set level. If the insulin delivery rate remains below this level for less than a specified percentage of time, the determined basal rate is lowered. Conversely, if the insulin delivery rate falls below the set level for more than the specified percentage of time, the determined basal rate is increased.

At step 905 of the flow diagram 900, the ambulatory medical device 220 may receive, from a sensor, a first biomarker level in the blood of the user 205. Examples of biomarkers include blood glucose levels and ketone levels in the user 205. An example of a sensor that may collect these biomarkers is the biochemical sensor 165, as shown in FIG. 1.

At step 910 of the flow diagram 900, the ambulatory medical device may determine a basal rate of endogenous glucose production based on one or more biomarker levels. For example, the ambulatory medical device 220 may determine the basal rate of endogenous glucose production based on the user's glucose level. In various embodiments, the ambulatory medical device may determine the basal rate based on the total daily dose of medicament or the user's body mass.

At step 915 of the flow diagram 900, the ambulatory medical device 220 may determine, based on the basal rate, an amount of medicament to transfer to the user. For example, if the ambulatory medical device determines that the user needs X amount of medicament and that the basal rate of endogenous production in the user is Y amount, the determination of the amount of medicament to be delivered may be X minus Y.

At step 920 of the flow diagram 900, the ambulatory medical device 220 may transmit a first instruction to the medical device to deliver the determined amount of medicament to the user. For example, the ambulatory medical device 220 may control a pump assembly that delivers the medicament through an infusion set. The instruction may specify an infusion rate based on the determined basal rate. The medicament may be delivered gradually over time or as a bolus dose.

At step 925 of the flow diagram 900, the ambulatory medical device 220 may receive a second biomarker level from a sensor. The second biomarker level may be a blood glucose concentration measured at a later time than the first biomarker level. In embodiments, the first biomarker level is a blood glucose concentration taken over a period of time and the second biomarker level is the blood glucose concentration taken over a later period of time.

At step 930 of the flow diagram 900, the ambulatory medical device may adjust the basal rate based on the first biomarker level and the second biomarker level. For example, after determining the basal rate at step 910, the basal rate may be adjusted based on the second biomarker level. In one example, the basal rate may be determined at step 910 using blood glucose levels in the user. At step 930, the basal rate may then be adjusted from the determined basal rate based on the second biomarker level. Accordingly, the first biomarker level and the second biomarker level may be used to adjust or determine the basal rate at step 930.

At step 935 of the flow diagram 900, the ambulatory medical device 220 may adjust the amount of medicament to transfer to the user based on the adjusted basal rate. Similar to step 915, the amount of medicament determined at step 935 is based on the amount needed by the user minus the basal rate of endogenous production in the user.

At step 940 of the flow diagram 900, the ambulatory medical device may transmit a second instruction to the medical device to deliver the amount of medicament to the user. For example, the controller may transmit an instruction to the pump assembly to deliver one or more medicaments to the user at a specified rate. The rate may be based on the amount of medicament determined in step 935. The amount of medicament determined in step 935 may be a total amount, a rate, or a combination of both. For example, the instruction may specify a total amount of medicament to be delivered at a set rate over a period of time.

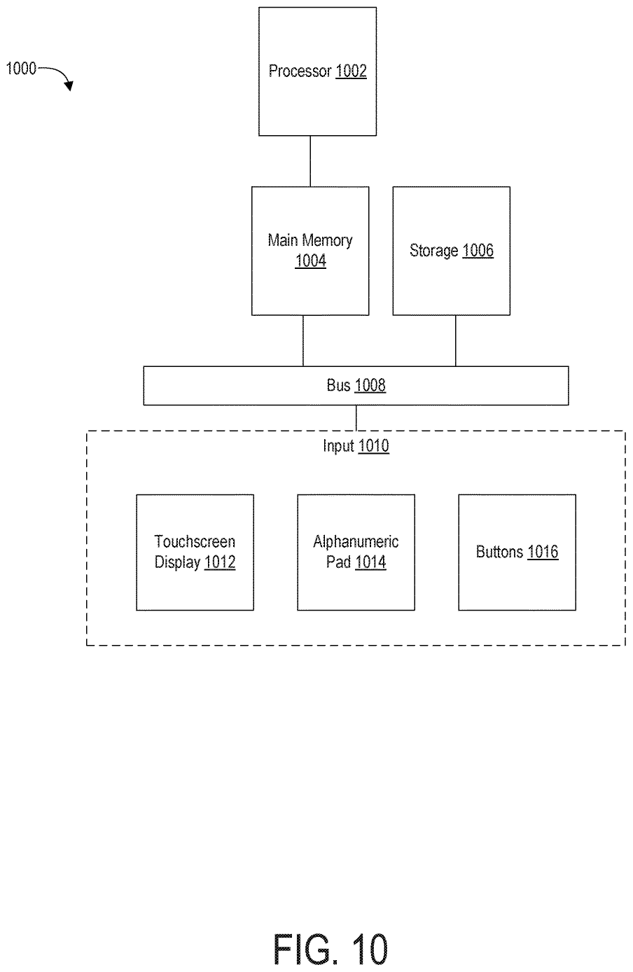

Referring to FIG. 10, FIG. 10 is a block diagram illustrating the computer system 1000 that may be implemented in the various embodiments in the described subject matter. The computer system 1000 includes a processor 1002, main memory 1004, storage 1006, a bus 1008, and input 1010. The processor 1002 may be one or more processors. The processor 1002 executes instructions that are communicated to the processor through the main memory 1004. The main memory 1004 feeds instructions to the processor 1002. The main memory 1004 is also connected to the bus 1008. The main memory 1004 may communicate with the other components of the computer system through the bus 1008. Instructions for the computer system 1000 are transmitted to the main memory 1004 through the bus 1008. Those instructions may be executed by the processor 1002. Executed instructions may be passed back to the main memory 1004 to be disseminated to other components of the computer system 1000. The storage 1006 may hold large amounts of data and retain that data while the computer system 1000 is unpowered. The storage 1006 is connected to the bus 1008 and can communicate data that the storage holds to the main memory 1004 through the bus 1008.

The processor 1002 may be any type of general purpose processor including, but not limited to a central processing unit (“CPU”), a graphics processing unit (“GPU”), a complex programmable logic device (“CPLD”), a field programmable gate array (“FPGA”), or an application-specific integrated circuit (“ASIC”). One embodiment of the computer system 1000 in the ambulatory medical device features a CPU as the processor 1002. However, embodiments may be envisioned for the computer system of the ambulatory medical device that incorporate other types of processors 1002.

The main memory 1004 can be any type of main memory that can communicate instructions to the processor 1002 and receive executed instructions from the processor 1002. Types of main memory 1004 include but are not limited to random access memory (“RAM”) and read only memory (“ROM”). In one embodiment, the computer system 1000 incorporates RAM as the form of main memory 1004 to communicate instructions to the processor 1002 and receive executed instructions from the processor 1002. Other embodiments may be envisioned that incorporate other types of main memory 1004 in the computer system 1000.

The storage 1006 can be any type of computer storage that can receive data, store data, and transmit data to the main memory 1004 via the bus 1008. Types of storage 1006 that can be used in the computer system 1000 include, but are not limited to, magnetic disk memory, optical disk memory, and flash memory. In one embodiment, flash memory is used as the storage 1006 in the computer system 1000 of the ambulatory medical device. Other embodiments that use other types of storage 1006 for the computer system 1000 may be envisioned.

The bus 1008 connects the internal components of the computer system 1000. The bus 1008 may include a multitude of wires that are connected to the components of the computer system 1000. The wires of the bus 1008 may differ based on the components of the computer system 1000 that the bus 1008 connects to. In various embodiments, the bus 1008 connects the processor 1002 to the main memory 1004. In various embodiments, the processor 1002 is directly connected to the main memory 1004.

The input 1010 of the computer system 1000 includes a touchscreen display 1012, an alphanumeric pad 1014, and buttons 1016. The touchscreen display 1012 both produces output and accepts input. The bus 1008 may be coupled to the touchscreen display 1012 to produce visual output. The touchscreen display 1012 may also accept input via capacitive touch, resistive touch, or other touch technology. The input surface of the touchscreen display 1012 can register the position of touches on the surface. Some types of touchscreen display 1012 can register multiple touches at once. The alphanumeric pad 1014 includes a multitude of keys with numerical, alphabetical, and symbol characters. Signals from the alphanumeric pad 1014 are communicated by the bus 1008 to the main memory 1004. Keys of the alphanumeric pad may be capacitive or mechanical. In some embodiments, the alphanumeric pad 1014 is displayed on the touchscreen display 1012. Buttons 1016, such as a meal announcement button, may be capacitive, mechanical, or other single input buttons. or other single input buttons.

A method for administering medicament from a medical device includes receiving, from a sensor, a first biomarker level in a user, determining a basal rate of endogenous glucose production based on one or more biomarker levels, determining, based on the basal rate, an amount of medicament to transfer to the user, transmitting a first instruction to the medical device to deliver the amount of medicament to the user, further receiving, from the sensor, a second biomarker level in the user, adjusting the basal rate based on the first biomarker level and the second biomarker level, adjusting the amount of medicament to transfer to the user based on the adjusted basal rate, and transmitting a second instruction to the medical device to deliver the amount of medicament to the user. The method may include determining an amount of un-metabolized medicament, and adjusting the amount of medicament may be further based on the amount of un-metabolized medicament. The biomarker level may be a glucose concentration, and the basal rate may be adjusted responsive to the glucose concentration being below or above a target threshold for greater or less than a time condition. The basal rate may be adjusted based on a mean glucose concentration and may be further adjusted based on a standard deviation of the glucose concentration. The method may further include determining a gain term for one or more biomarkers in the user, and adjusting the basal rate may be further based on the gain term.

A system for administering medicament to a user includes an ambulatory medication administration device comprising a reservoir configured to store an amount of medicament, a pump configured to dispense the medicament from the reservoir to a user carrying the ambulatory medication administration device, a sensor configured to collect a biomarker concentration in the user, and a controller. The controller is configured to receive, from a sensor, a first biomarker level in a user, determine a basal rate of endogenous glucose production based on one or more biomarker levels, determine, based on the basal rate, an amount of medicament to transfer to the user, transmit a first instruction to the medical device to deliver the amount of medicament to the user, further receive, from the sensor, a second biomarker level in the user, adjust the basal rate based on the first biomarker level and the second biomarker level, adjust the amount of medicament to transfer to the user based on the adjusted basal rate, and transmit a second instruction to the medical device to deliver the amount of medicament to the user. The biomarker level may be a glucose concentration over a period of time. The basal rate may be adjusted responsive to the glucose concentration being below or above a target threshold for greater or less than a time condition. The basal rate may be adjusted based on a mean glucose concentration and may be further adjusted based on a standard deviation of the glucose concentration. The system may include determining a gain term for one or more biomarkers in the user, and adjusting the basal rate may be further based on the gain term.

An ambulatory medical device includes a reservoir configured to store an amount of medicament, a pump configured to dispense the medicament from the reservoir to a user carrying the ambulatory medication administration device, a sensor configured to collect a first biomarker concentration in the user, and a controller. The controller is configured to receive, from a sensor, a biomarker level in a user, determine a basal rate of endogenous glucose production based on one or more biomarker levels, determine, based on the basal rate, an amount of medicament to transfer to the user, transmit a first instruction to the medical device to deliver the amount of medicament to the user, further receive, from the sensor, a second biomarker level in the user, adjust the basal rate based on the first biomarker level and the second biomarker level, adjust the amount of medicament to transfer to the user based on the adjusted basal rate, and transmit a second instruction to the medical device to deliver the amount of medicament to the user. The controller may be further configured to determine an amount of un-metabolized medicament, and the controller may adjust the amount of medicament further based on the amount of un-metabolized medicament. The biomarker level may be a glucose concentration. The basal rate may be adjusted responsive to the glucose concentration being below or above a target threshold for greater or less than a time condition.

Many variations may be made to the embodiments described herein. All variations, including combinations of embodiments, are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

1. A method for administering medicament from a medical device, the method comprising:
receiving, from a sensor, a first biomarker level in a user;
determining a basal rate of endogenous glucose production based on the first biomarker level;
determining, based on the basal rate, an amount of medicament to transfer to the user;
transmitting a first instruction to the medical device to deliver the amount of medicament to the user;
further receiving, from the sensor, a second biomarker level in the user;
adjusting the basal rate based on the first biomarker level and the second biomarker level;
adjusting the amount of medicament to transfer to the user based on the adjusted basal rate; and
transmitting a second instruction to the medical device to deliver the amount of medicament to the user.

2. The method of claim 1, further comprising determining an amount of un-metabolized medicament; and
wherein adjusting the amount of medicament is further based on the amount of un-metabolized medicament.

3. The method of claim 1, wherein the biomarker level is a glucose concentration.

4. The method of claim 3, wherein the basal rate is adjusted responsive to the glucose concentration being below a target threshold for greater or less than a time condition.

5. The method of claim 3, wherein the basal rate is adjusted responsive to the glucose concentration being above a target threshold for greater or less than a time condition.

6. The method of claim 3, wherein the basal rate is adjusted based on a mean glucose concentration.

7. The method of claim 6, wherein the basal rate is further adjusted based on a standard deviation of the glucose concentration.

8. The method of claim 1, further comprising determining a gain term for one or more biomarkers in the user; and
wherein adjusting the basal rate is further based on the gain term.

9. A system for administering medicament to a user, the system comprising:
an ambulatory medication administration device, the ambulatory medication administration comprising:
a reservoir configured to store an amount of medicament;
a pump configured to dispense the medicament from the reservoir to a user carrying the ambulatory medication administration device;
a sensor configured to collect a biomarker concentration in the user; and
a controller, the controller configured to:
receive, from a sensor, a first biomarker level in a user;
determine a basal rate of endogenous glucose production based on the first biomarker level;
determine, based on the basal rate, an amount of medicament to transfer to the user;
transmit a first instruction to the medical device to deliver the amount of medicament to the user;
further receive, from the sensor, a second biomarker level in the user;
adjust the basal rate based on the first biomarker level and the second biomarker level;
adjust the amount of medicament to transfer to the user based on the adjusted basal rate; and
transmit a second instruction to the medical device to deliver the amount of medicament to the user.

10. The system of claim 9, wherein the biomarker level is a glucose concentration over a period of time.

11. The system of claim 10, wherein the basal rate is adjusted responsive to the glucose concentration being below a target threshold for greater or less than a time condition.

12. The system of claim 10, wherein the basal rate is adjusted responsive to the glucose concentration being above a target threshold for greater or less than a time condition.

13. The system of claim 10, wherein the basal rate is adjusted based on a mean glucose concentration.

14. The system of claim 13, wherein the basal rate is further adjusted based on a standard deviation of the glucose concentration.

15. The system of claim 9, further comprising determining a gain term for one or more biomarkers in the user; and
wherein adjusting the basal rate is further based on the gain term.

16. An ambulatory medical device, the ambulatory medical device comprising:
a reservoir configured to store an amount of medicament;
a pump configured to dispense the medicament from the reservoir to a user carrying the ambulatory medical administration device;
a sensor configured to collect a first biomarker concentration in the user; and
a controller, the controller configured to:
receive, from a sensor, biomarker level in a user;
determine a basal rate of endogenous glucose production based on the first biomarker level;
determine, based on the basal rate, an amount of medicament to transfer to the user;
transmit a first instruction to the medical device to deliver the amount of medicament to the user;
further receive, from the sensor, a second biomarker level in the user;
adjust the basal rate based on the first biomarker level and the second biomarker level;
adjust the amount of medicament to transfer to the user based on the adjusted basal rate; and
transmit a second instruction to the medical device to deliver the amount of medicament to the user.

17. The ambulatory medical device of claim 16, wherein the controller is further configured to determine an amount of un-metabolized medicament; and
wherein the controller configured to adjust the amount of medicament is further based on the amount of un-metabolized medicament.

18. The ambulatory medical device of claim 16, wherein the biomarker level is a glucose concentration.

19. The ambulatory medical device of claim 18, wherein the basal rate is adjusted responsive to the glucose concentration being below a target threshold for greater or less than a time condition.

20. The ambulatory medical device of claim 18, wherein the basal rate is adjusted responsive to the glucose concentration being above a target threshold for greater or less than a time condition.
